(19) United States
(12) Patent Application Publication
(10) Pub. No.: US 2015/0174016 A1
(43) Pub. Date: Jun. 25, 2015

(54) MOISTURIZING AND GENTLE EXFOLIATING SKIN CLEANSING COMPOSITION

(71) Applicant: The Dial Corporation, Scottsdale, AZ (US)

(72) Inventor: Kanani Guerra, Phoenix, AZ (US)

(73) Assignee: The Dial Corporation, Scottsdale, AZ (US)

(21) Appl. No.: 14/139,908

(22) Filed: Dec. 24, 2013

(51) Int. Cl. A61K 8/04; A61Q 19/00; A61Q 19/10

(52) U.S. Cl. CPC A61K 8/044; A61Q 19/10; A61Q 19/007

(57) ABSTRACT

Methods and apparatus are provided for an exfoliating skin cleansing composition which is both gentle and moisturizing. The exfoliating skin cleansing composition includes a number of surfactants. The exfoliating skin cleansing composition further includes a number of suspending aides. The exfoliating skin cleansing composition also includes a number of exfoliating particles. The exfoliating skin cleansing composition also includes an aqueous medium in which the number of surfactants, the number of suspending aides and the number of exfoliating particles are distributed.

### FIELD OF THE INVENTION

The present invention generally relates to an exfoliating skin cleansing composition, and more particularly relates to an exfoliating skin cleansing composition with a low solids content which is both moisturizing and gentle.

### BACKGROUND OF THE INVENTION

Exfoliating skin cleansing compositions may contain large amounts of surfactants, suspending aides, or both, in order to suspend exfoliating particles in the skin cleansing composition. The inclusion of a large quantity of surfactants or suspending aides may make an exfoliating skin cleansing composition prone to cause skin dryness, and may result in poor moisturization characteristics.

Accordingly, it is desirable to have an exfoliating skin cleansing composition which has reduced amounts of both surfactants and suspending aides, while stably suspending exfoliating particles. In addition, it is desirable to maintain favorable skin feel properties, as well as to have such an exfoliating skin cleansing composition be both moisturizing and gentle. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A transparent exfoliating skin cleansing composition comprises from 1.0 to 9.0 weight percent of a number of surfactants, from 0.50 to 4.0 weight percent of a number of suspending aides, from 0.25 to 4.0 weight percent of a number of exfoliating particles, and an aqueous medium in which the number of surfactants, the number of suspending aides, and the number of exfoliating particles are distributed.

An exfoliating skin cleansing product comprises a container, and an exfoliating skin cleansing composition, which skin cleansing composition comprises from 1.0 to 9.0 weight percent of a number of surfactants, from 0.50 to 4.0 weight percent of a number of suspending aides, from 0.25 to 4.0 weight percent of a number of exfoliating particles, and an aqueous medium in which the number of surfactants, the number of suspending aides and the number of exfoliating particles are distributed.

An exfoliating skin cleansing composition comprises from 7.5 to 9.0 weight percent of a number of surfactants, from 1.5 to 3.0 weight percent of a number of suspending aides, from 0.25 to 1.5 weight percent of a number of exfoliating agents, and an aqueous medium in which the number of surfactants, the number of suspending aides and the number of exfoliating particles are suspended. The non-aqueous components of the exfoliating skin cleansing composition are less than 15 weight percent of the exfoliating skin cleansing composition, and the exfoliating skin cleansing composition decreases the conductance of skin by an amount less than 4.0 microSiemens.

### BRIEF DESCRIPTION OF THE DRAWINGS

The present invention will hereinafter be described in conjunction with the following drawing figures, wherein like numerals denote like elements, and

FIG. 1 is a diagram of an exemplary container dispensing an exfoliating skin cleansing composition, according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

An exfoliating skin cleansing composition may contain sufficient surfactants and suspending aides so as to stably suspend the exfoliating particles. However, an amount of surfactants and suspending aides used to stably suspend the exfoliating skin cleansing compositions may cause dryness of the skin, or generally lack moisturization properties, which moisturization properties may lead to consumer acceptance of the exfoliating skin cleansing composition.

The consumer acceptance of an exfoliating skin cleansing composition may be based on its application aesthetics. These application aesthetics may be a function of the exfoliating skin cleansing composition's lather, skin feel, and other rheological properties, as well as the visibility of any exfoliating particles. Visible exfoliating particles may improve the consumer's impression that the exfoliating skin cleansing composition is acting to effectively exfoliate the skin being cleansed. Additionally, the tactile sensation of the exfoliating particles may further contribute to the impression that the exfoliating skin cleansing composition is effectively exfoliating the skin being cleansed.

Visible exfoliating particles may contribute to consumer acceptance of an exfoliating skin cleansing composition, as the suspension of the exfoliating particles in the solution may be visible before a consumer purchases or tests an exfoliating skin cleansing composition. Thus, the transparency of an exfoliating skin cleansing composition may be valuable to its consumer acceptance.

In addition to transparency of the exfoliating skin cleansing composition, the ability of an exfoliating skin cleansing composition to also be moisturizing and gentle, while retaining its exfoliating properties, may further enhance consumer acceptance. If an exfoliating skin cleansing composition is both moisturizing and gentle, then it may be used repeatedly and regularly, providing the regular sensation of effective exfoliation, without resulting in the sensation of excessively dry skin, or loss of efficacy of the barrier function of the skin.

Accordingly, the principles described herein provide a composition which allows for effective suspension of a number of exfoliating particles, using a reduced amount of surfactants and suspending aides, and which may result in an exfoliating skin cleansing composition which is both gentle and moisturizing. The principles described herein may also provide for an exfoliating skin cleansing composition which possesses desirable application aesthetics, such as lather, skin feel, effective exfoliation and other rheological properties.

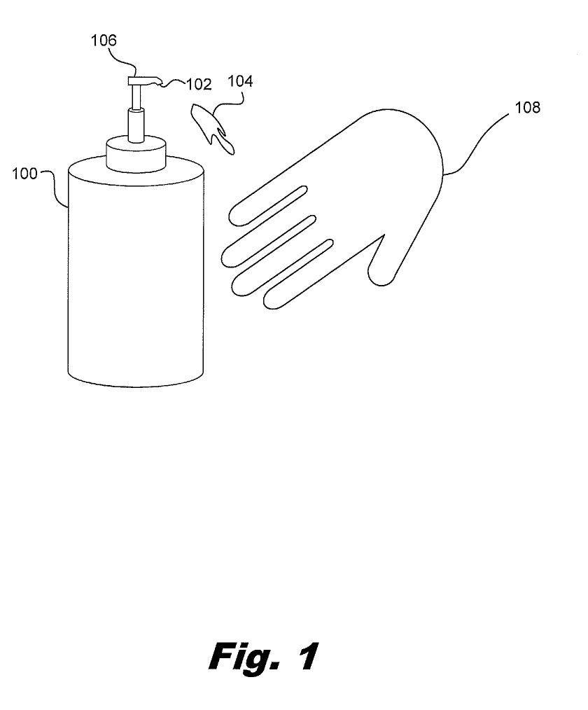

Turning now to the figures, FIG. 1 is a diagram of an exemplary container (100), dispensing an exfoliating skin cleansing composition (104) according to the principles described herein. The container (100) may be equipped with an opening (102) capable of dispensing the exfoliating skin cleansing composition (104). The opening (102) may dispense the exfoliating skin cleansing composition (104) by a plunger type mechanism (106), or another appropriate dispenser. The opening (102) may be configured so as to dispense the exfoliating skin cleansing composition (104) onto an area of the skin. For example, the opening (102) may be configured so as to dispense the exfoliating skin cleansing composition (104) onto a hand (108) of a user. The container (100) shown in FIG. 1 is exemplary and does not represent all types of dispensers or shapes of containers which may be used to dispense the exfoliating skin cleansing composition (104). Another type of dispensing container (100) may include a simple opening allowing for the dispensing of the exfoliating skin cleansing composition (104), which container (100) may have a cap.

The exfoliating skin cleansing composition (104) may include a number of surfactants. A surfactant may have a hydrophobic end and a hydrophilic end. The hydrophobic end may allow the surfactant to interact with uncharged molecules, such as oils. The hydrophilic end may facilitate the interaction of the molecule with charged or polar molecules, such as water. The hydrophilic end may be used to classify surfactants, which may be anionic, cationic, nonionic, or amphoteric. Anionic surfactants may have a negatively charged hydrophilic end, which may be present as a sulfate, sulfonate, carboxylate, phosphate, or the like; anionic surfactants may be sensitive to water hardness. Cationic surfactants may be those that have a positively charged hydrophilic end, such as a quaternary amine. Nonionic surfactants may have a hydrophilic end which may be charge neutral, such as an ethoxylate, glycoside, or poly-ol; such surfactants may not be sensitive to water hardness. Amphoteric or zwitterionic surfactants may have both a positive and negative charge on their hydrophilic ends, such as amine oxides, sultaines, or betaines. In some examples, the number of surfactants may include lauramidopropyl betaine, lauryl betaine, lauryl sultaine, lauryl sulfate, lauryl sulfonate, cocamidopropyl betaine, myristyl betaine, myristyl sultaine, myristyl sulfate, myristyl sulfonate, cetyl betaine, cetyl sultaine, cetyl sulfate, cetyl sulfonate, stearyl betaine, stearyl sultaine, stearyl sulfate, stearyl sulfonate, or combinations thereof. While specific reference has been made to certain surfactants, the number of surfactants in the exfoliating skin cleansing composition (104) may include any type, combination or mixture of surfactants. For example, the number of surfactants may include a single anionic surfactant. Another example may have the number of surfactants be a blend of nonionic and amphoteric surfactants. The number of surfactants may comprise between 1.0 and 9.0 weight percent of the exfoliating skin cleansing composition (104). For example, the number of surfactants may comprise between 7.5 and 9.0 weight percent of the exfoliating skin cleansing composition (104), and more specifically between 8.0 and 9.0 weight percent of the exfoliating skin cleansing composition (104).

The exfoliating skin cleansing composition (104) may include a number of suspending aides. A suspending aide may be a molecule which assists in the suspension of a solid particle in a solution. A suspending aide may thicken the composition, providing a gel-like material in which a solid may be suspended. A suspending aide may be a polymer or copolymer, which may be capable of suspending a number of solid particles in solution. Suspending aides may comprise polymers of acrylamide, acrylate, or saccharides, which may be subsequently modified by conjugation of remaining functional groups with additional components, such as alkyl ethers. Examples of suspending aides include acrylate/C10-30 alkyl acrylate crosspolymer, acrylates copolymer, acrylates crosspolymer, acrylates crosspolymer-3, acrylates crosspolymer-4, carbomer, cellulose gum, methylcellulose, methyl hydroxyethylcellulose, polyacrylate crosspolymer-4, polyacrylate crosspolymer-6, polyacrylate crosspolymer-11, polyacrylate crosspolymer-14, polyacrylic acid, poly C10-30 alkyl acrylate, xanthan gum, guar gum, or combinations thereof. While specific reference has been made to certain suspending aides, the number of suspending aides in the exfoliating skin cleansing composition (104) may include any type, combination or mixture of suspending aides. For example, the number of suspending aides may include a polysaccharide, such as xanthan gum, and an acrylate polymer, such as polyacrylate crosspolymer-11. Another example may have the number of suspending aides be a single polysaccharide suspending aide. The number of suspending aides may comprise between 0.5 and 4.0 weight percent of the exfoliating skin cleansing composition (104). For example, the number of suspending aides may comprise between 1.5 and 3.0 weight percent of the exfoliating skin cleansing composition (104), and more specifically between 2.0 and 2.5 weight percent of the exfoliating skin cleansing composition (104).

The exfoliating skin cleansing composition (104) may include a number of exfoliating particles. Exfoliating particles may be solids suspended in the exfoliating skin cleansing composition (104), which may act as an abrasive when the exfoliating skin cleansing composition (104) is used to cleanse skin. The abrasive may remove dead skin cells from the surface of the skin. Removal of the dead skin cells from the surface of the skin may allow improved access and deeper penetration of the surfactants and any moisturizer which may be present in the exfoliating skin cleansing composition. This may improve both the feel and appearance of skin. The number of exfoliating particles may comprise between 0.25 and 4.0 weight percent of the exfoliating skin cleansing composition (104). For example, the exfoliating particles may comprise between 0.25 and 1.5 weight percent of the exfoliating skin cleansing composition (104), and more specifically between 0.5 and 1.0 weight percent of the exfoliating skin cleansing composition (104).

Examples of exfoliating skin particles which may be included in an exfoliating skin cleansing composition (104) include actinidia chinensis (kiwi) seed, alumina, aluminum iron silicates, aluminum silicate, amethyst powder, amorphophallus konjac root powder, arachis hypogaea (peanut) flour, attapulgite, avena sativa (oat) bran, avena sativa (oat) kernel flour, avena sativa (oat) kernel meal, bambusa arundinacea stem powder, calcium carbonate, calcium phosphate, calcium pyrophosphate, calcium sulfate, carya illinoensis (pecan) shell powder, chalk, chitin, citrus tangerina (tangerine) peel, cocos nucifera (coconut) shell powder, colloidal oatmeal, conchiolin powder, coral powder, corylus avellana (hazel) shell powder, diamond powder, diatomaceous earth, dicalcium phosphate, dicalcium phosphate dihydrate, dolomite, egg shell powder, eijitsu, elguea clay, emerald, fragaria vesca (strawberry) seed, fuller's earth, glycine soja (soybean) flour, helianthus annuus (sunflower) seed meal, hordeum distichon (barley) seed flour, hordeum vulgare powder, hordeum vulgare seed flour, hydrated silica, hydroxyapatite, illite, juglans mandshurica (walnut) shell powder, juglans regia (walnut) shell powder, kaolin, kurumi kaku, lauryl acrylate/VA crosspolymer, lithothamnium calcarum powder, lithoammium corallioides powder, loess, luffa cylindrica fruit, magnesium potassium fluorosilicate, magnesium sodium fluorosilicate, magnesium trisilicate, melaleuca alternifolia leaf powder, microcrystalline cellulose, montmorillonite, moroccan lava clay, mother of pearl, myristyl betaine, oenothera biennis (evening primrose) seed, olea europaea (olive) fruit, olea europaea (olive) husk powder, olea europaea (olive) seed powder, oryza sativa (rice) bran, oryza sativa (rice) germ powder, oubaku, oyster shell powder, papaver so niferum seed, perlite, persea gratissima (avocado) fruit powder, phaseolus radiatus seed starch, platinum powder, polyethylene, potassium undecylenoyl glutamate, prunus amygdalus dulcis (sweet almond) seed meal, prunus amygdalus dulcis (sweet almond) shell powder, prunus armeniaca (apricot) seed powder, prunus mume fruit, prunus persica (peach) seed powder, pumice, quartz, rubus idaeus (raspberry) seed, salt mine mud, sand, sea salt, secale cereale (rye) seed flour, silica, sodium bicarbonate, sodium hydroxypropyl starch phosphate, sodium magnesium fluorosilicate, sodium silicoaluminate, symphytum officinale leaf powder, talc, theobroma cacao (cocoa) shell powder, tin oxide, titanium oxynitride, topaz, touki, tricalcium phosphate, triticum vulgare (wheat) bran, triticum vulgare (wheat) germ powder, triticum vulgare (wheat) kernel flour, triticum vulgare (wheat) starch, vaccinium angustifolium (blueberry) seed, vaccinium macrocarpon (cranberry) seed, volcanic ash, wood powder, yokuinin, zea mays (corn) cob meal, zea mays (corn) cob powder, zea mays (corn) kernel meal, zea mays (corn) seed flour, zea mays (corn) starch, zirconium silicate, or combinations thereof. While specific reference has been made to certain exfoliating particles, the number of exfoliating particles may include any type, combination, or mixture of exfoliating particles. For example, the number of exfoliating particles may be prunus persica seed powder. In another example, the number of exfoliating particles may include dolomite, emerald, platinum powder, microcrystalline cellulose, and zea mays corn meal.

The exfoliating skin cleansing composition (104) may include an aqueous medium in which the number of surfactants, the number of suspending aides, the number of exfoliating particles, and any other components are distributed. The aqueous medium may be water, and may comprise less than 97 weight percent of the exfoliating skin cleansing composition (104). For example, the aqueous medium may comprise between 85 and 88 weight percent of the exfoliating skin cleansing composition (104).

The exfoliating skin cleansing composition (104) may also include a number of other components. These components may include an antimicrobial active ingredient, a moisturizer, a salt, a preservative, a dye and a thickening agent.

The exfoliating skin cleansing composition (104) may include an antimicrobial active ingredient. An antimicrobial active ingredient may be an ingredient which kills bacteria. Examples of antimicrobial active ingredients may include triclosan, benezethonium chloride, benzalkonium chloride, antibiotics which inhibit the 70S ribosome, and combinations thereof.

The exfoliating skin cleansing composition (104) may include a moisturizer. Moisturizers may be chemical agents which cause the skin to be softer. Moisturizers may act by reducing evaporation from the surface of the skin. Humectants may be used as moisturizers. Oils may be used as moisturizers. Examples of moisturizers may include lipids, sterols, and combinations thereof. Examples of lipids may include oleic acid, palmitic acid, myristic acid, lauric acid, triglycerides, phospholipids, sphingolipids, prenol lipids, saccharolipids, polyketide lipids, and combinations thereof. The hydrocarbon chain of a lipid may be either saturated or unsaturated, and unsaturated chains may be either cis or trans. Examples of sterols may include campesterol, cholesterol, ergosterol, sitosterol, and stigmasterol. While specific reference has been made to certain moisturizers, a moisturizer may include any type, combination or mixture of moisturizers. For example, a moisturizer may include trimystirin. Another example of a moisturizer may include lauric acid, distearyl phosphatidylinositol, 1,3-dipalmitoyl-2-oleoyl-glycerol, and tristearoyl glycerol. The moisturizer may comprise between 1.0 and 2.0 weight percent of the exfoliating skin cleansing composition (104). For example, the moisturizer may comprise between 1.0 and 1.25 weight percent of the exfoliating skin cleansing composition (104).

The exfoliating skin cleansing composition (104) may include a salt. Salts may be composed of a cation and an anion, which have no net charge, and may form a crystalline structure in the absence of water. The anion and cation of a salt may dissociate in the presence of water, and may modulate the ionic strength of an aqueous solution. Examples of salts may include sodium chloride, sodium bromide, sodium iodide, calcium chloride, calcium bromide, calcium iodide, lithium chloride, lithium bromide, lithium iodide, beryllium chloride, beryllium bromide, beryllium iodide, potassium chloride, potassium bromide, potassium iodide, magnesium chloride, magnesium bromide, magnesium iodide, magnesium oxide, copper(I) chloride, copper(I) bromide, copper(II) chloride, copper (II) bromide, iron(II) chloride, iron(II) bromide, iron(III) chloride, iron(III) bromide, strontium chloride, strontium bromide, platinum(II) chloride, platinum(II) bromide, platinum(IV) chloride, platinum(IV) bromide, and the like. While specific reference has been made to certain salts, a salt may include any type, combination or mixture of salts. For example, a salt may include sodium chloride. Another example of a salt may include gold(I) chloride, copper(I) chloride and potassium bromide. The salt may comprise less than 2.0 weight percent of the exfoliating skin cleansing composition (104). For example, the salt may comprise between 0.5 and 1.0 weight percent of the exfoliating skin cleansing composition (104).

The exfoliating skin cleansing composition (104) may also include a preservative, a dye, a thickening agent, or combinations thereof. These ingredients may be added in order to protect the exfoliating skin cleansing composition (104) from damage due to light or chemically-induced deterioration, to improve consumer acceptance by introducing favorable colors, or to modulate the viscosity of the exfoliating skin cleansing composition (104).

The exfoliating skin cleansing composition (104) may be transparent. This may allow the suspension of exfoliating particles in the exfoliating skin cleansing composition (104) to be visible, which may increase consumer appeal. The visibility of the exfoliating particles in the exfoliating skin cleansing composition may ensure consumer awareness that the skin cleansing composition is exfoliating, as well as increase consumer confidence that the exfoliating skin cleansing composition (104) may cause effective exfoliation.

The exfoliating skin cleansing composition (104) may include an aqueous component, and a number of non-aqueous components. The aqueous component may comprise water, which may be an aqueous medium in which the non-aqueous components are distributed. The aqueous component may comprise less than 97 weight percent of the exfoliating skin cleansing composition (104). The non-aqueous component may include all other ingredients. For example, the non-aqueous component may comprise the number of surfactants, the number of suspending aides, the number of exfoliating particles. In another example, the non-aqueous components may also comprise a moisturizer, a salt, and a preservative. The non-aqueous components may comprise less than 20 weight percent of the composition. For example, the non-aqueous components may comprise less than 15 weight percent of the composition.

A soap which is moisturizing, or which may be more moisturizing than water, may have increased consumer appeal as a result of its ability to increase the moisture content of the skin surface, which may make the skin surface appear softer and healthier. Water may be used as a threshold, and if a skin cleansing composition demonstrates a statistical improvement to moisturization properties when compared to water, the skin cleansing composition may be said to be moisturizing. The moisturizing effect of a skin cleansing composition may be measured by skin conductance, corneometry or visual evaluation. Each of skin conductance, comeometry and visual evaluation may compare a baseline measurement with a number of measurements taken after treatment with a skin cleansing composition, which may be exfoliating. The number of measurements taken after treatment with a skin cleansing composition, which may be exfoliating, may be taken at regular intervals, and may be used as a basis of comparison between a number of skin cleansing compositions.

Skin conductance may be a method of measuring the moisture content of skin. Comparing values before treatment with a skin cleansing composition and at regular intervals following treatment may allow determination of whether a skin cleansing composition is moisturizing, or whether a skin cleansing composition removes moisture from skin. Such a treatment may include washing with a skin cleansing composition. Test washing with a skin cleansing composition may be conducted on the lower leg, and skin conductance measurements may be made at the wash site. Temperature and humidity may affect skin conductance measurements, and so may be held constant from the baseline measurement to subsequent measurements. Skin conductance may be measured in units of microSiemens, and may be the reciprocal of skin resistance. Skin conductance measurements may be presented as the difference from the baseline measurement.

Table (1) below may present skin conductance measurements comparing the exfoliating skin cleansing composition (104) to both water and a moisturizing skin cleansing composition, which may serve as a non-exfoliating positive control, and be so marked in Table (1). The values presented in Table (1) may indicate the difference from the baseline measurement, and may represent the mean deviation from the baseline measurement for a study group of seventeen subjects. Positive numbers may indicate an increase in the moisture content of the skin; negative numbers may indicate a decrease in the moisture content of the skin. Values may be presented as the difference from the baseline measurement, in units of microSiemens.

TABLE (1)

| Skin Cleansing Composition | 0.5 hrs. | 1 hr. | 2 hrs. | 3 hrs. | Overall |
|---|---|---|---|---|---|
| Exfoliating Skin Cleansing | −2.75 | −3.68 | −2.32 | −2.28 | −2.76 |
| Composition (104) | | | | | |
| Water | −5.15 | −5.40 | −3.90 | −3.74 | −4.54 |
| Positive Control | −1.16 | −2.28 | −0.82 | 0.12 | −1.04 |

As indicated in Table (1), the exfoliating skin cleansing composition (104) may be more moisturizing than water. Since the measurements at each time show a larger negative number for water than for the exfoliating skin cleansing composition, and negative numbers may indicate a decrease in the moisture content of the skin, the exfoliating skin cleansing composition (104) may be said to be moisturizing.

Corneometry may be another way to measure the moisture content of skin. Corneometry may measure the capacitance of the skin, which may be a dielectric medium. The measured capacitance may be used to determine the dielectric constant of the skin, which may be converted to an arbitrary hydration unit, which may indicate a moisture content of the skin. Corneometry may measure the capacitance and hydration of the stratum corneum, which may be an outer layer of the epidermis. The stratum corneum may serve as a barrier, and measurements of its hydration may be used as a proxy for its ability to function as a barrier.

Table (2) below may present corneometry measurements comparing the exfoliating skin cleansing composition (104) to both water and a moisturizing skin cleansing composition, which may serve as a non-exfoliating positive control, and be so marked in Table (2). The values presented in Table (2) may be presented as the difference from the baseline measurement, and may represent the mean deviation from the baseline measurement for a study group of seventeen subjects. Positive numbers may indicate an increase in the moisture content of the skin; negative numbers may indicate a decrease in the moisture content of the skin. Values may be presented as the mean difference from the baseline measurement, in corneometry units.

TABLE (2)

| Skin Cleansing Composition | 0.5 hrs. | 1 hr. | 2 hrs. | 3 hrs. | Overall |
|---|---|---|---|---|---|
| Exfoliating Skin Cleansing | −1.66 | −1.41 | −1.15 | −1.09 | −1.33 |
| Composition (104) | | | | | |
| Water | −2.85 | −3.28 | −2.65 | −2.35 | −2.78 |
| Positive Control | 0.10 | −0.60 | 0.49 | 0.85 | 0.21 |

As indicated in Table (2), the exfoliating skin cleansing composition (104) may be more moisturizing than water. Since the measurements at each time show a larger negative number for water than for the exfoliating skin cleansing composition, and negative numbers may indicate a decrease in the moisture content of the skin, the exfoliating skin cleansing composition (104) may be said to be moisturizing.

Moisture content of skin may also be determined by visual evaluations. Similar to both skin conductance and corneometry, visual evaluation may be relative to a baseline evaluation. Negative values for visual evaluation may indicate an increase in the moisture content of the skin, which may be measured as a decrease in the visual evaluation of skin dryness. Visual evaluations may be conducted by an evaluator using a 3× diopter lens illuminated magnifying glass. A baseline evaluation may be conducted, as well as subsequent evaluations at 0.5, 1, 2, and 3 hours after application. Evaluations may be assigned a numerical value, or ‘grade,’ according to the scale presented in Table (3).

TABLE (3)

| Grade | Dryness |
|---|---|
| 0.0 | No visible dryness |
| 0.5 | Perceptible dryness with fine white lines |
| 1.0 | Fine dry lines and a white powdery appearance, which may |
| | be accompanied by some uplifting flakes on less than |
| | 30% of the evaluation site |
| 1.5 | Uniform flaking which covers 30-50% of the evaluation site |
| 2.0 | Uniform, marked flaking covering more than 50% of the |
| | evaluation site |
| 2.5 | Slight to moderate scaling |
| 3.0 | Moderate to severe scaling, which may be accompanied |
| | by uplifting scales |
| 3.5 | Severe scaling, which may be accompanied by slight fissuring |
| 4.0 | Severe scaling and fissuring |

Table (4) below may present visual evaluations of skin dryness comparing the exfoliating skin cleansing composition (104) to both water and a moisturizing skin cleansing composition, which may serve as a non-exfoliating positive control, and be so marked in Table (4). Values presented in Table (4) may indicate the difference from the baseline measurement, and may represent the mean deviation from the baseline measurement for a study group of seventeen subjects. Positive numbers may indicate an increase in dryness of the skin, which may correspond to a decrease in moisture content of the skin, and negative numbers may indicate a decrease in dryness of the skin, which may correspond to an increase in moisture content of the skin. Values may be presented as the average difference in the grade from the baseline measurement, according to the grading shown in Table (3). As indicated in Table (4), the exfoliating skin cleansing composition (104) may be at least as moisturizing as water. Since the measurements at each time are more negative for the exfoliating skin cleansing composition than those for water, and negative numbers may indicate a decrease in skin dryness, which may correspond to an increase in skin moisturization, the exfoliating skin cleansing composition (104) may be said to be moisturizing.

TABLE (4)

| Skin Cleansing Composition | 0.5 hrs. | 1 hr. | 2 hrs. | 3 hrs. |
|---|---|---|---|---|
| Exfoliating Skin Cleansing Composition | −0.91 | −0.62 | −0.41 | −0.47 |
| (104) | | | | |
| Water | −0.38 | −0.21 | −0.21 | −0.26 |
| Positive Control | −0.79 | −0.68 | −0.50 | −0.50 |

Water may remove natural moisturizing factor from the skin surface, which may result in decreased moisture content in the skin surface. This decrease in moisture content of skin may be evaluated by skin conductance, corneometry, or visual evaluation. Application of water to the skin may temporarily increase the appearance of moisture content on the skin surface by visual evaluation.

The gentleness of an exfoliating skin cleansing composition (104) may affect its consumer appeal. As an exfoliating skin cleansing composition (104) may include exfoliating particles, numerous washes may cause irritation of the skin. If an exfoliating skin cleansing composition (104) does not cause irritation of the skin, then its gentleness properties may increase consumer appeal because the exfoliating skin cleansing composition may be used for regular exfoliation without discoloration or other signs of harsh treatment to the surface of the skin.

Exaggerated washing may be used to measure the gentleness of a skin cleansing composition. Exaggerated washing may expose skin to a large number of washings in a fixed time period, for example 35 washes over four days, which may be conducted with ten washings per day for the first three days and five washings on the final day. Washings may be conducted in sets of five washings; each washing in the set may be separated by approximately five minutes; each set of washings may be separated by at least two hours. Each wash may consist of a 30 second application of 1.5 milliliters of skin cleansing composition. The results of exaggerated washing may be assessed by visual erythema assessments, visual dryness assessments, and transepidermal water loss assessment of skin barrier (TEWL) scores. Visual erythema assessments and visual dryness assessments may be conducted before each set of washings, as well as two hours following the final washing. This may provide for a baseline measurement, as well as measurements before the 6^{th}, 11^{th}, 16^{th}, 21^{st}, 26^{th}, and 31^{st} washing, and the final measurement after the 35^{th} washing. TEWL measurements may be taken both prior to the first washing and two hours following the final washing. Water may be used as a threshold, and if a skin cleansing composition demonstrates comparable or improved hydration scores, and produces no additional dryness or erythema when compared to water, the skin cleansing composition may be said to be gentle.

Visual erythema assessments and visual dryness assessments may be conducted by an evaluator according to the grading system outlined in Table (5). Evaluations of visual erythema and visual dryness may occur at regular intervals throughout the exaggerated washing study, for example every five washes. The grading system in Table (5) ranges from 0.0 to 3.0, using increments of 0.5.

TABLE (5)

| Grade | Erythema | Dryness |
|---|---|---|
| 0.0 | None | None |
| 0.5 | Perceptible | Perceptible, whiteness in the lines of the skin |
| 1.0 | Mild, slight | Slight flaking or uplifting of flakes, which may be |
| | | accompanied by a patchy or powdered appearance |
| 1.5 | Slight to | Slight to moderate flaking or uplifting of flakes, |
| | moderate | which may be uniform on the evaluation site |
| 2.0 | Moderate, | Moderate flaking or uplifting of flakes, which |
| | confluent | may be uniform on the evaluation site, and |
| | | may also be acompied by slight scaling |
| 2.5 | Moderate to | Moderate to severe flaking or uplifting of flakes, |
| | marked | which may be accompanied by moderate scaling |
| 3.0 | Marked, | Severe flaking or scaling, uplifting of scales, |
| | prominent | which may be accompanied by slight fissuring |

Visual erythema assessments may comprise a visual assessment of any increased redness in the skin, which may be indicative of irritation to the skin. A visual assessment of erythema may be conducted at regular intervals throughout the exaggerated washing, for example every five washes. Table (6) may show visual erythema assessments taken throughout an exaggerated washing study. Table (6) may present visual erythema assessments for the dorsal hand, finger webbing and volar forearm. In Table (6), measurement 1 may correspond to the measurement taken before washing six; measurement 2 to the measurement taken before washing eleven; measurement 3 to the measurement taken before washing sixteen; measurement 4 to the measurement taken before washing twenty-one; measurement 5 to the measurement taken before washing twenty-five; measurement 6 to the measurement taken before washing thirty-one; and measurement 7 to the measurement taken two hours after the thirty-fifth washing. Visual assessments of erythema may be presented as the average difference from the baseline measurement in the study group of 36 subjects, and may be measured according to the grading system presented in Table (5). Positive values may indicate an increase in visual assessment of erythema, and negative values may indicate a decrease in visual assessment of erythema.

TABLE (6)

| Measurement | Dorsal Hand | Finger Webbing | Volar Forearm |
|---|---|---|---|
| 1 | 0.03 | 0.0 | 0.0 |
| 2 | −0.03 | 0.0 | 0.0 |
| 3 | 0.0 | 0.0 | 0.0 |
| 4 | −0.03 | 0.0 | 0.0 |
| 5 | 0.06 | 0.0 | 0.0 |
| 6 | 0.0 | 0.0 | 0.0 |
| 7 | 0.03 | 0.0 | 0.0 |

Visual dryness assessments may comprise a visual assessment of the dryness of the skin surface, which may indicate the effect that exaggerated washing has on the moisture content of the skin surface. A visual dryness assessment may be conducted at regular intervals throughout the exaggerated washing, for example every five washes. Table (7) may show visual dryness assessments taken throughout an exaggerated washing study. Table (7) may present visual dryness assessments for the dorsal hand, finger webbing and volar forearm. In Table (7), measurement 1 to the measurement taken before washing six; measurement 2 to the measurement taken before washing eleven; measurement 3 to the measurement taken before washing sixteen; measurement 4 to the measurement taken before washing twenty-one; measurement 5 to the measurement taken before washing twenty-five; measurement 6 to the measurement taken before washing thirty-one; and measurement 7 to the measurement taken two hours after the thirty-fifth washing. Visual assessments of dryness may be presented as the average difference from the baseline measurement in the study group of 36 subjects, and may be measured according to the grading system presented in Table (5). Positive values may indicate an increase in visual assessment of dryness, and negative values may indicate a decrease in visual assessment of dryness.

TABLE (7)

| Measurement | Dorsal Hand | Finger Webbing | Volar Forearm |
|---|---|---|---|
| 1 | 0.06 | −0.09 | 0.16 |
| 2 | 0.09 | −0.03 | 0.06 |
| 3 | 0.13 | −0.06 | 0.16 |
| 4 | 0.06 | −0.16 | 0.06 |
| 5 | 0.28 | −0.09 | 0.22 |
| 6 | −0.06 | −0.16 | 0.03 |
| 7 | 0.03 | −0.16 | 0.19 |

Transepidermal water loss assessment of skin barrier (TEWL) may be an assessment of the skin barrier functionality which measures the amount of water that passes through the epidermis to the surrounding atmosphere, for example by diffusion or evaporation. TEWL measurements may be assigned a score, which may be presented as the difference from the baseline measurement or score. TEWL measurements may be taken in duplicate, and may include a baseline measurement and a second measurement two hours following the final washing of an exaggerated washing study. In the study which produced the data shown in Tables (6) and (7), the average TEWL measurement may be 2.2, which may be the difference from the baseline measurement, and may be less than similar measurements conducted when water was the only treatment.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A transparent exfoliating skin cleansing composition, comprising:
from 1.0 to 9.0 weight percent of a number of surfactants;
from 0.50 to 4.0 weight percent of a number of suspending aides;
from 0.25 to 4.0 weight percent of a number of exfoliating particles; and
an aqueous medium in which the number of surfactants, the number of suspending aides and the number of exfoliating particles are distributed.

2. The exfoliating skin cleansing composition of claim 1, wherein a combined weight of the non-aqueous components is less than 20% of the weight of the exfoliating skin cleansing composition.

3. The exfoliating skin cleansing composition of claim 1, wherein a combined weight of the non-aqueous components is less than 15% of the weight of the exfoliating skin cleansing composition.

4. The exfoliating skin cleansing composition of claim 1, wherein the number of surfactants comprises anionic surfactants, amphoteric surfactants, or combinations thereof.

5. The exfoliating skin cleansing composition of claim 1, wherein the number of suspending aides comprises between 1.50 and 3.0 weight percent of the exfoliating skin cleansing composition.

6. The exfoliating, skin cleansing composition of claim 1, in which the exfoliating skin cleansing composition causes decreases in an amount less than 4.0 microSiemens to skin conductance measurements of skin which has been treated with the exfoliating skin cleansing composition.

7. The exfoliating skin cleansing composition of claim 1, in which the exfoliating skin cleansing composition causes average changes in an amount less than 0.20 to visual assessments of erythema, according to an incremented grading system, during exaggerated washing.

8. The exfoliating skin cleansing composition of claim 1, in which the exfoliating skin cleansing composition causes average changes in an amount less than 0.3 to visual assessments of skin dryness, according to an incremented grading system, during exaggerated washing.

9. The exfoliating skin cleansing composition of claim 1, wherein the pH of the exfoliating skin cleansing composition is between 5.5 and 8.0.

10. The exfoliating skin cleansing composition of claim 1, wherein the viscosity of the exfoliating skin cleansing composition is between 2,000 and 20,000 centipoise.

11. The exfoliating skin cleansing composition of claim 1, further comprising a moisturizer, a salt, a preservative, a dye, a thickening agent, an active antimicrobial ingredient, or combinations thereof.

12. An exfoliating skin cleansing product, comprising:
a container; and
an exfoliating skin cleansing composition, comprising:
from 1.0 to 9.0 weight percent of a number of surfactants;
from 0.50 to 4.0 weight percent of a number of suspending aides;
from 0.25 to 4.0 weight percent of a number of exfoliating particles; and
an aqueous medium in which the number of surfactants, the number of suspending aides, and the number of exfoliating particles are distributed.

13. The exfoliating skin cleansing product of claim 11, wherein the exfoliating skin cleansing composition is transparent.

14. The exfoliating skin cleansing product of claim 11, wherein a combined weight of the non-aqueous components of the exfoliating skin cleansing composition is less than 15% of the weight of the exfoliating skin cleansing composition.

15. The exfoliating skin cleansing product of claim 11, wherein the number of surfactants may include anionic surfactants, amphoteric surfactants, or combinations thereof.

16. The exfoliating skin cleansing product of claim 11, wherein the exfoliating skin cleansing composition further comprises a moisturizer, a salt, a preservative, a dye, a thickening agent, an active antimicrobial ingredient, or combinations thereof.

17. An exfoliating skin cleansing composition, comprising:
from 7.5 to 9.0 weight percent of a number of surfactants;
from 1.50 to 3.0 weight percent of a number of suspending aides;
from 0.25 to 1.5 weight percent of a number of exfoliating agents;
an aqueous medium in which the number of surfactants, the number of suspending aides, and the number of exfoliating particles are distributed; wherein
the non-aqueous components of the exfoliating skin cleansing composition are less than 15 weight percent of the exfoliating skin cleansing composition; and
the exfoliating skin cleansing composition decreases the conductance of skin in an amount less than 4.0 microSiemens.

18. The exfoliating skin cleansing composition of claim 17, wherein the number of surfactants comprises anionic surfactants, amphoteric surfactants, or combinations thereof.

19. The exfoliating skin cleansing composition of claim 17, wherein the exfoliating skin cleansing composition is transparent.

20. The exfoliating skin cleansing composition of claim 17, further comprising a moisturizer, a salt, a preservative, a dye, a thickening agent, an active antimicrobial ingredient, or combinations thereof.
